(19) United States
(12) Patent Application Publication
(10) Pub. No.: US 2020/0080999 A1
(43) Pub. Date: Mar. 12, 2020

(54) METHOD AND DEVICE FOR EVALUATING IMMUNE CELLS USING MAGNETIC PARTICLES

(71) Applicant: UNIST(ULSAN NATIONAL INSTITUTE OF SCIENCE AND TECHNOLOGY), Ulsan (KR)

(72) Inventors: Joo Hun Kang, Ulsan (KR); Se Yong Kwon, Ulsan (KR); Min Seok Lee, Ulsan (KR)

(73) Assignee: UNIST(ULSAN NATIONAL INSTITUTE OF SCIENCE AND TECHNOLOGY), Ulsan (KR)

(21) Appl. No.: 16/684,532

(22) Filed: Nov. 14, 2019

Related U.S. Application Data
(63) Continuation-in-part of application No. PCT/KR2018/005684, filed on May 17, 2018.

(30) Foreign Application Priority Data
May 17, 2017 (KR) 10-2017-0061247
May 17, 2018 (KR) 10-2018-0056541
May 17, 2018 (KR) 10-2018-0056542

(51) Int. Cl. G01N 33/543; B01L 3/00

(52) U.S. Cl. CPC G01N 33/54333; B01L 3/508; B01L 2300/0816; B01L 2300/06; G01N 2800/24

(57) ABSTRACT

Provided are a method of and apparatus for evaluating immune cells using magnetic particles. According to an aspect of the method, magnetic particles may be used to measure interaction of the magnetic particles with immune cells, thereby diagnosing or evaluating a degree of activation of immune cells of a subject or immune-related diseases of the subject. Further, according to an aspect of the apparatus, a phenomenon of interaction of magnetic particles and activated immune cells via endocytosis may be used to collect magnetic particle-immune cell complexes resulting from the interaction with the magnetic particles by applying a magnetic field, thereby effectively isolating the activated immune cells in a short period of time.

### CROSS-REFERENCE TO RELATED APPLICATION

This is a continuation-in-part of International Application No. PCT/KR2018/005684, filed May 17, 2018, which in turn claims priority to Korean Patent Application No. 10-2017-0061247, filed May 17, 2017, Korean Patent Application No. 10-2018-0056541, filed May 17, 2018 and Korean Patent Application No. 10-2018-0056542, filed May 17, 2018, which applications are incorporated herein in their entireties.

### TECHNICAL FIELD

The present disclosure relates to a method of and apparatus for evaluating immune cells using magnetic particles.

### BACKGROUND ART

Immune cells are cells involved in specific recognition/binding, non-specific binding, or endocytosis of immunogens (e.g., exogenous immunogen and/or endogenous immunogen) in the immune system of a living body of an organism (e.g., animals such as mammals, birds, fish, etc.).

Immune cells activate the immune system by direct contact between cells or by recognizing water-soluble molecules derived from a microorganism, substances derived from injured cells of a host, etc. Receptors for these molecules are present in the immune cells. When the receptors recognize the molecules, the immune cells produce cytokines, interferons, and chemokines to cause immune responses. Therefore, it is possible to diagnose a variety of infectious diseases or immune-related diseases by using activation of the immune cells.

Generally, to diagnose infectious diseases, a cell culture method or a gene detection method to identify a microorganism which is a cause of infection has been used. Also, to diagnose immune-related diseases, a method of detecting the presence of antibodies in blood has been used. However, there is a problem in that these methods take a long time of one day or more and require much cost, and the procedures are complicated.

Accordingly, there is a need for a method of and apparatus for diagnosing a variety of infectious diseases, immune-related diseases, and immune disorders by simply evaluating the activation of immune cells at low cost and in a short period of time.

### DESCRIPTION OF EMBODIMENTS

### Technical Problem

An aspect provides a method of evaluating a degree of activation of immune cells of a subject by using magnetic particles and endocytosis of immune cells.

Another aspect provides an apparatus for isolating activated immune cells.

Embodiments of the present disclosure, which have been created to evaluate the activation of immune cells in a quick and simple manner, provide an apparatus and a method capable of effectively isolating activated immune cells and evaluating a degree of activation of the immune cells, in which a phenomenon of interaction of magnetic particles and activated immune cells via endocytosis is used to collect magnetic particle-immune cell complexes resulting from the interaction with the magnetic particles by applying a magnetic field.

### Solution to Problem

An aspect provides a method of evaluating a degree of activation of immune cells of a subject, the method including contacting magnetic particles with a sample separated from the subject, wherein the sample includes the immune cells; isolating immune cells that interacted with the magnetic particles from the immune cells in the sample by applying a magnetic field to a reaction product obtained by the contacting; and detecting the isolated immune cells that interacted with the magnetic particles.

The method of evaluating the degree of activation of immune cells of the subject may include contacting magnetic particles with the sample separated from the subject, wherein the sample includes the immune cells.

The immune cells may interact with the magnetic particles by contact between the magnetic particles and the immune cells included in the sample separated from the subject.

As used herein, the term “interaction” refers to a phenomenon in which magnetic particles adhere to, enter, are invaginated by, or are trapped in the interior or exterior of immune cells by endocytosis of immune cells. The endocytosis, also called intracellular uptake, collectively refers to a phenomenon in which cells ingest substances. Specifically, the endocytosis may include phagocytosis or pinocytosis.

The endocytosis frequently occurs by immune cells activated by infection, etc., rather than inactivated immune cells, That is, the degree of activation of immune cells is proportional to incidence of endocytosis, and the immune cells that interacted with magnetic particles may be evaluated as activated immune cells.

Due to the interaction, a magnetic particle-immune cell complex may be formed.

For example, the contacting of magnetic particles with immune cells included in the sample separated from the subject may include culturing the sample and the magnetic particles. The culturing may be performed under common conditions using a medium which is commonly used in culturing body fluids such as blood, etc. or immune cells.

The contacting may be performed under conditions sufficient for interaction of the immune cells and the magnetic particles. In an embodiment, the contacting may be performed at about 0° C. to about 40° C., about 30° C. to about 40° C., or 35° C. to about 40° C., or for about 0.1 sec to about 1 hr, about 1 sec to about 1 hr, about 30 sec to about 1 hr, or about 1 min to about 1 hr, but is not limited thereto. The contacting time is shorter than a time taken to evaluate activation of immune cells by a method which is generally used, and according to an aspect, the degree of activation of the immune cells may be evaluated in a short time.

The term “subject” refers to an object to be evaluated for the degree of activation of immune cells. The subject may be one or more selected from primates such as human, monkey, etc., rodents such as rats, mice, etc., artiodactyla such as horse, cow, pig, sheep, goat, etc., mammals such as, Equidae, Canidae, Felidae, etc., birds, fish, etc.

As used herein, the term “sample” may be a biological sample. The biological sample may be a body fluid (e.g., blood, plasma, serum, saliva, sputum, or urine), an organ, a tissue, a fraction, or a cell isolated from a mammal including a human, but is not limited thereto. The sample may also include an extract from the biological sample, for example, an antibody, a protein, etc. from a biological fluid (e.g., blood or urine). The sample is not limited, as long as it includes immune cells. For example, the sample may include blood, urine, feces, saliva, lymph, cerebrospinal fluid, synovial fluid, cystic fluid, ascites, interstitial fluid, or ocular fluid.

As used herein, the term “immune cells” may be all kinds of cells involved in specific recognition: binding, non-specific binding, or endocytosis of immunogens (e.g., exogenous immunogen and/or endogenous immunogen) in the immune system of a living body of an organism (e.g., animals such as mammals, birds, fish, etc.). Specifically, the immune cells may include one or more selected from the group consisting of neutrophils, eosinophils, basophils, monocytes, lymphocytes, Cooper cells, microglia, alveolar macrophages, connective tissue macrophages (histiocyte), macrophages such as dendritic cells, mast cells, B cells, T cells, natural killer cells (NK cells), immune cell-derived cell lines, and stem cell-derived immune cells. The “immune cell-derived cell lines” refer to cell lines derived from immune cells, and the “stem cell-derived immune cells” refer to immune cells differentiated from stem cells by a technique known in the art.

The immune cells may be labeled with a detectable label. The label may be all kinds of labels (small molecule compounds, proteins, or poly/oligopeptides, etc.) which may be detected by a common method, and for example, the label may be one or more selected from the group consisting of fluorescent substances, luminescent substances, etc.

As used herein, the term “magnetic particles” may include any particles as long as they may have magnetic properties and may be readily absorbed by cells without cytotoxicity. Specifically, the magnetic particles may include one or more magnetic atoms selected from the group consisting of iron (Fe), nickel (Ni), cobalt (Co), manganese (Mn), bismuth (Bi), zinc (Zn), strontium (Sr), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), Erbium (Er), thulium (Tm), ytterbium (Yb), ruthenium (Lu), copper (Cu), silver (Ag), gold (Au), cadmium (Cd), mercury (Hg), aluminum (Al), gallium (Ga), indium (In), thallium (TI), calcium (Ca), Barium (Ba), radium (Ra), platinum (Pt), and lead (Pd).

The magnetic particles may be oxidized or surface-modified. Specifically, iron may be oxidized to form iron oxide. The surface modification may be surface modification by metals, surface modification by functional groups such as a carboxyl group or an amine group, surface modification by proteins including streptavidin, avidin, immunoglobulins, C-reactive protein (CRP), and opsonins such as mannose-binding lectins, or complement proteins, surface modification by carbohydrates, surface modification by polymers, or surface modification by lipids, but is not limited thereto. The modification may stabilize the magnetic particles. Further, the modification may improve interaction between magnetic particles and activated immune cells.

The magnetic particles may be prepared by a known method or purchased from a commercially available source.

The magnetic particles may be used as they are or in a state in which the magnetic particles are dispersed or suspended in an appropriate solvent (e.g., buffers (PBS, saline, Tris-buffered saline, etc.), etc.), but is not limited thereto.

The magnetic particles may have a small particle size, and thus individual particles have a single magnetic domain. Therefore, the magnetic particles have magnetic properties only in the presence of an external magnetic field to exhibit superparamagnetism. Superparamagnetic magnetic particles may be simply and quickly separated by applying an external magnetic field. The separation of the magnetic particles by applying a magnetic field is not influenced by environmental conditions such as pH, temperature, ions, etc., and thus excellent in terms of stability and sensitivity.

The magnetic particles may be selected from all kinds of particles having magnetic properties and having such a particle size that the particles may interact with immune cells, for example, may adhere to, may enter, may be invaginated by, or may be trapped in immune cells. For example, the magnetic particles may be magnetic particles having an average particle size of about 1 nm to about 30,000 nm, about 10 nm to about 30,000 nm, about 50 nm to about 30,000 nm, about 100 nm to about 30,000 nm, about 200 nm to about 30,000 nm, about 300 nm to about 30,000 nm, about 400 nm to about 30,000 nm, about 500 nm to about 30,000 nm, about 1 nm to about 20,000 nm, about 10 nm to about 20,000 nm, about 50 nm to about 20,000 nm, about 100 nm to about 20,000 nm, about 200 nm to about 20,000 nm, about 300 nm to about 20,000 nm, about 400 nm to about 20,000 nm, about 500 nm to about 20,000 nm, about 1 nm to about 10,000 nm, about 10 nm to about 10,000 nm, about 50 nm to about 10,000 nm, about 100 nm to about 10,000 nm, about 200 nm to about 10,000 nm, about 300 nm to about 10,000 nm, about 400 nm to about 10,000 nm, about 500 nm to about 10,000 nm, about 1 nm to about 5,000 nm, about 10 nm to about 5,000 nm, about 50 nm to about 5,000 nm, about 100 nm to about 5,000 nm, about 200 nm to about 5,000 nm, about 300 nm to about 5,000 nm, about 400 nm to about 5,000 nm, about 500 nm to about 5,000 nm, 1 nm to about 1,000 nm, about 10 nm to about 1,000 nm, about 50 nm to about 1,000 nm, about 100 nm to about 1,000 nm, about 200 nm to about 1,000 nm, about 300 nm to about 1,000 nm, about 400 nm to about 1,000 nm, about 500 nm to about 1,000 nm, about 1 nm to about 500 nm, about 10 nm to about 500 nm, about 50 nm to about 500 nm, about 100 nm to about 500 nm, about 200 nm to about 500 nm, about 300 nm to about 500 nm, or about 400 nm to about 500 nm, but are not limited thereto.

The method of evaluating the degree of activation of immune cells of the subject may include isolating immune cells that interacted with the magnetic particles from the immune cells included in the sample by applying a magnetic field to a reaction product obtained by the contacting; and detecting the isolated immune cells that interacted with the magnetic particles,

The magnetic field may be formed by any common method, for example, by using a magnet, such as an electromagnet by electromagnetic induction, a permanent magnet, etc. The magnet may be one or more, and may be applied in various arrays such as serial array, parallel array, circular array, alternative array, etc. The application of the magnetic field is not influenced by environmental conditions such as pH, temperature, ions, etc., and thus excellent in terms of stability.

The reaction product obtained by the contacting may include immune cells that interacted with the magnetic particles, immune cells that did not interact with the magnetic particles, the magnetic particles, or a mixture thereof. By applying a magnetic field to the reaction product, magnetic particle-immune cell complexes gather around the magnetic field, and among the immune cells included in the sample, immune cells that interacted with magnetic particles may be separated from immune cells that did not interact with magnetic particles.

The applying of the magnetic field may be performed for a sufficient time to gather the magnetic particles and the immune cells that interacted with the magnetic particles around the magnetic field. For example, the applying of the magnetic field may be performed for about 0.1 sec to about 1 hr, about 1 sec to about 1 hr, about 30 sec to about 1 hr, or about 1 min to about 1 hr. The time for applying the magnetic field is generally shorter than a time taken to evaluate activation of immune cells by a method which is generally used, and according to an aspect, the degree of activation of immune cells may be evaluated in a short time.

The detecting is to detect the isolated immune cells that interacted with the magnetic particles. In one embodiment, the immune cells may be labeled with a detectable label, and the immune cells that interacted with the magnetic particles may be identified by detecting the label.

The method of evaluating the degree of activation of the immune cells of the subject may further include comparing a level of the detected immune cells (activated immune cells) that interacted with the magnetic particles with a level of immune cells in the sample or comparing the level of the immune cells (activated immune cells) that interacted with the magnetic particles in the sample separated from the subject with a level of immune cells (activated immune cells) that interacted with the magnetic particles in a sample separated from a normal subject.

In comparing the level of the detected immune cells that interacted with the magnetic particles with the level of the immune cells in the sample, the immune cells that interacted with the magnetic particles are activated immune cells, and the immune cells in the sample include the immune cells that interacted with the magnetic particles and the immune cells that did not interact with the magnetic particles. The level of the immune cells in the sample may be evaluated by detecting immune cells which are labeled with a detectable label in any process before isolating the immune cells that interacted with the magnetic particles. A ratio of the activated immune cells to the entire immune cells may be measured by comparing the level of the detected immune cells that interacted with the magnetic particles with the level of the immune cells in the sample. The ratio of the activated immune cells to the entire immune cells is compared with a ratio in a normal subject, and when the ratios of the activated immune cells in the two subjects are different from each other, the subject may be diagnosed as having or being at risk of suffering from infectious diseases or immune-related diseases. For example, when the ratio of the activated immune cells to the entire immune cells in the subject is higher than the ratio of the activated immune cells in the normal subject, the subject may be diagnosed as being exposed to an immune stimulation (immune activation) state, a hyperimmune state, or infectious diseases. For example, when the ratio of the activated immune cells to the entire immune cells in the subject is lower than the ratio of the activated immune cells in the normal subject, the subject may be diagnosed as being at an immune inactivation state or an immune deficiency state.

In comparing the level of the immune cells that interacted with the magnetic particles in the sample separated from the subject with the level of the immune cells that interacted with the magnetic particles in the sample separated from the normal subject, the number of the immune cells that interacted with the magnetic particles may be the number of activated immune cells, and thus the number of activated immune cells of the subject which is a target of evaluation may be compared with the number of activated immune cells of the normal subject.

In the method, the “degree of activation of immune cells” may refer to a degree of endocytosis of immune cells which is increased or decreased by infection or immune disorder.

Another aspect provides a method of diagnosing immune-related diseases, the method including contacting magnetic particles with a sample separated from a subject, wherein the sample includes the immune cells; isolating immune cells that interacted with the magnetic particles from the immune cells included in the sample by applying a magnetic field to a reaction product obtained by the contacting; and detecting the isolated immune cells that interacted with the magnetic particles.

Each of the processes is the same as described above.

In the method, the “immune-related diseases” may be any disease caused by stimulation of the immune system (i.e., induction of an immune activation state or an immune inactivation state), immune stimulation (immune activation), a hyperimmune state, immune inactivation, or immune deficiency, and the immune-related diseases may be, for example, one or more selected from the group consisting of systemic or local infection (e.g., early infection or chronic infection) by viruses, bacteria, fungi, mycete, etc., inflammation (e.g., acute inflammation or chronic inflammation), sepsis, cancer, cancer metastasis, autoimmune diseases, and cardiovascular diseases (arteriosclerosis, stroke, etc.). More specifically, the immune-related diseases may include diseases related to or caused by an immune stimulation (immune activation) state or an immune disorder (i.e., hyperimmune) state such as systemic or local infection, acute inflammation, sepsis, autoimmune diseases, cardiovascular diseases (arteriosclerosis, stroke, etc.); or diseases related to or caused by an immune disorder (i.e., immune inactivation or immune deficiency) state such as chronic infection, chronic inflammation, cancer, cancer metastasis, etc.

Still another aspect provides an apparatus for separating activated immune cells, the apparatus including a chamber for storing a sample including immune cells and magnetic particles; and a magnetic field-forming portion which is disposed to apply a magnetic field around the chamber, wherein activated immune cells, among the immune cells in the sample, interact with magnetic particles to form magnetic particle-immune cell complexes, and the magnetic particle-immune cell complexes are collected around a magnetic field formed by the magnetic field-forming portion.

Hereinafter, the present disclosure will be described in detail by embodiments only for illustrative purposes with reference to the accompanying drawings. The following embodiments are intended to specify the present disclosure and not to limit or restrict the scope of the present disclosure. Those easily inferred from detailed description and embodiments by a person skilled in the art to which the present disclosure pertains should be construed as being included in the scope of present disclosure.

The term “consist of” or “include”, as used herein, should not be construed as essentially including all several elements or several steps described in the specification, but the terms may be construed as not including some of the elements or steps or as including additional element or steps.

Further, the terms such as “portion”, “module”, as used herein, denote a unit that processes at least one function or operation.

Although the terms including an ordinal number such as “first”, “second”, etc., as used herein, may be used for describing various subjects, the subjects are not restricted by the terms. The terms are used merely for the purpose to distinguish a subject from the other subjects.

FIG. 1 shows an apparatus 1 for isolating activated immune cells according to an embodiment.

Referring to FIG. 1, the apparatus 1 for isolating the activated immune cells may include a chamber 110 and a magnetic field-forming portion 130.

The chamber is a device that includes a space in which an experiment subject is placed during an experiment. A sample which is an evaluation subject of the apparatus for isolating the activated immune cells and magnetic particles may be placed in the chamber. The space in the chamber may maintain conditions regarding temperature, humidity, light, gas compositions, etc., under which cells may be allowed to grow and maintain growth.

The chamber is not limited to any form, as long as it contains the sample and magnetic particles or the sample and magnetic particles are movable therein, but the chamber may be in a form of a tube, a channel, a well, a droplet, or any combination thereof.

As used herein, the term “channel” means a path through which a fluid travels, and for example, a channel extending along a planar flow path (e.g., a channel of a twisted or spiral planar pattern), a non-planar flow path (e.g., a helical three-dimensional channel), or a microfluidic channel.

The magnetic field-forming portion may be any hardware or electric circuit applying a magnetic field. For example, the magnetic field-forming portion may include at least one magnet, and the magnet may be a magnet such as an electromagnet by electromagnetic induction, a permanent magnet, etc. The magnet may be disposed on one side of the chamber, e.g., on the upper, lower, or side surface of the chamber, or in various arrays such as serial array, parallel array, circular array, alternative array, etc. such that the magnet may apply the magnetic field around the chamber.

In an embodiment, immune cells activated or not activated by infections or immune responses exist in the immune cells included in the sample. Under this state, the sample including the immune cells react with magnetic particles before or after placing them in the chamber, and the activated immune cells interact with the magnetic particles via endocytosis, resulting in formation of magnetic particle-immune cell complexes. Due to the magnetic particles, the formed magnetic particle-immune cell complexes gather around the chamber by the magnetic field which is formed by the magnetic field-forming portion and as a result, the activated immune cells may be simply and effectively separated in a short time.

Referring to FIG. 2, the apparatus 1 for isolating the activated immune cells may further include an inlet 120 or an outlet 140.

The inlet is a device for moving the sample and the magnetic particles to the chamber. The inlet may be connected to an end part of the chamber. A plurality of inlets, for example, 2, 3, 4, 5, or more may be used, Alternatively, the inlet may be part of the chamber.

The inlet may be connected to an end part of the chamber.

The outlet is a device for discharging the remaining sample, magnetic particles, or immune cells, except for magnetic particle-immune cell complexes which are formed by interaction of the magnetic particles and the activated immune cells among the immune cells included in the sample. When the remaining sample except for magnetic particle-immune cell complexes is discharged via the outlet and the sample and the magnetic particles are continuously injected into the inlet, a large amount of activated immune cells in the sample may be effectively collected.

The outlet may be connected to another end part of the chamber.

FIG. 3 illustrates the apparatus 1 for isolating the activated immune cells according to an embodiment.

Referring to FIG. 3, the apparatus 1 for isolating the activated immune cells may include the chamber 110, the inlet 120, the magnetic field-forming portion 130, and a detecting portion 150.

The detecting portion is a device for detecting the magnetic particle-immune cell complexes which are collected and immobilized by the magnetic field. In the detecting, for example, when the immune cells included in the magnetic particle-immune cell complexes are labeled with a detectable label such as a fluorescent material, the detecting portion may include a fluorescence microscope for detecting the fluorescent material.

In an embodiment, immune cells activated or not activated by infections or immune responses exist in the immune cells included in the sample. Under this state, the sample including the immune cells react with magnetic particles before placing them in the chamber, and the activated immune cells interact with the magnetic particles via endocytosis, resulting in formation of magnetic particle-immune cell complexes. Due to the magnetic particles, the formed magnetic particle-immune cell complexes gather at the bottom or top of the chamber by the magnetic field formed by the magnetic field-forming portion, when the magnetic field is formed below or above the chamber in the direction of gravity. If the magnetic field-forming portion exists on the chamber, the magnetic particle-immune cell complexes float to the top of the chamber, and the number of inactivated immune cells that settle down at the bottom of the chamber is counted by the detecting portion such as a fluorescent microscope, and compared with the number of the entire immune cells to calculate the number of the activated immune cells.

FIG. 4 illustrates an apparatus for isolating the activated immune cells according to an embodiment.

Referring to FIG. 4, an apparatus 4 for isolating the activated immune cells may include a chamber 410, a separating portion 411, inlets including a first inlet 421 and a second inlet 422, and a magnetic field-forming portion 430.

Further, the apparatus may include a plurality of outlets.

The chamber 410 and the magnetic field-forming portion 430 respectively perform the same functions as the chamber 110 and the magnetic field-forming portion 130 of FIG. 1, and their descriptions will be omitted.

The first inlet and the second inlet may be connected to an end part of the chamber. A sample, a sample analog composed of components similar to the sample, e.g., a dilution of the sample, magnetic particles, or a mixture thereof may be injected into the first inlet or the second inlet, respectively.

The separating portion is a device for separating the magnetic particle-immune cell complexes from the mixture of the sample and magnetic particles injected into the inlet. Further, when a plurality of inlets are used, the separating portion is a device for separating injected materials from each of a plurality of separating portions in one chamber.

Further, the separating portion may have a channel array structure. Since the separating portion has this structure, the magnetic particle-immune cell complexes are less influenced by a flow direction from the inlet to the outlet, and are influenced only by a force in the direction of the magnetic field, thereby effectively separating the magnetic particle-immune cell complexes from the separating portion having a limited length.

The magnetic field-forming portion may be disposed on one surface of the chamber so as to apply a magnetic field.

In an embodiment, a sample dilution is injected via the first inlet, and the sample and the magnetic particles are injected via the second inlet. Since the first inlet and the second inlet are connected to an end part of the chamber, the sample dilution injected via the first inlet and the sample and the magnetic particles injected via the second inlet may be contained or may move in one chamber with the separating portion as a boundary. Here, when the magnetic field-forming portion is disposed on a surface of the chamber, specifically, disposed such that the magnetic field is applied around the chamber containing the sample dilution which is injected via the first inlet, magnetic particle-immune cell complexes formed from the sample and the magnetic particles injected via the second inlet may gather around the magnetic field through the channel array included in the separating portion. Here, this function may be also performed even without the channel array included in the separating portion. As the dilution and the sample continue to flow toward the outlet, the sample is discharged through the outlet in a direction away from the magnetic field, and only the magnetic particle-immune cell complexes in the sample are pulled toward the dilution, and discharged through the outlet in a direction close to the magnetic field. Therefore, it is possible to easily separate and collect the magnetic particle-immune cell complexes from the sample without any separate equipment.

### ADVANTAGEOUS EFFECTS OF DISCLOSURE

According to a method of an aspect, magnetic particles may be used to measure interaction of the magnetic particles with immune cells, thereby diagnosing or evaluating a degree of activation of immune cells of a subject or immune-related diseases. Further, according to an apparatus of another aspect, a phenomenon of interaction of magnetic particles and activated immune cells via endocytosis may be used to collect magnetic particle-immune cell complexes resulting from the interaction with the magnetic particles by applying a magnetic field, thereby effectively isolating the activated immune cells in a short period of time.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates an apparatus for isolating activated immune cells according to an embodiment;

FIG. 2 illustrates the apparatus for isolating activated immune cells according to an embodiment;

FIG. 3 illustrates the apparatus for isolating activated immune cells according to an embodiment;

FIG. 4 illustrates the apparatus for isolating activated immune cells according to an embodiment;

FIG. 5 shows results of isolating activated immune cells from a control group (healthy sample) and E.coli-infected blood (infection model) in vitro through the apparatus for isolating activated immune cells according to an embodiment;

FIG. 6 shows a photograph (left) of immune cells before contacting blood of a control rat with magnetic particles in vivo and a photograph (right) of remaining immune cells except for immune cells that interacted with the magnetic particles by contacting, through the apparatus for isolating activated immune cells according to an embodiment; and

FIG. 7 shows a photograph (left) of immune cells before contacting blood of an E.coli-infected rat with magnetic particles in vivo and a photograph (right) of remaining immune cells except for immune cells that interacted with the magnetic particles by contacting, through the apparatus for isolating activated immune cells according to an embodiment;

FIG. 8 is a graph showing white blood cells detected at each time point of infection of rats with E. coil, through the apparatus for isolating activated immune cells according to an embodiment;

FIG. 9 is an immunofluorescence (IF) image of identifying the presence of E.coli in an organ of a rat;

FIG. 10 is a graph showing white blood cells detected in a breast cancer mouse model, through the apparatus for isolating activated immune cells according to an embodiment;

FIG. 11 is an image of fluorescence-stained white blood cells, through the apparatus for isolating activated immune cells according to an embodiment;

FIG. 12 is a graph showing neutrophils in white blood cells which were isolated using magnetic particles, through the apparatus for isolating activated immune cells according to an embodiment;

FIG. 13 is a fluorescence microscopy image of white blood cells reacted with mannose binding lectin (MBL)-immobilized magnetic particles; and

FIG. 14 is a graph showing a comparison of endocytosis between a diabetic rat model and a normal rat, through the apparatus for isolating activated immune cells according to an embodiment.

### MODE OF DISCLOSURE

Hereinafter, the present disclosure will be described in more detail with reference to embodiments. However, these embodiments are for illustrative purposes only, and the scope of the present disclosure is not intended to be limited thereby. It will be apparent to those skilled in the art that modifications may be made to the following embodiments without departing from the essential spirit of the present disclosure.

### Example 1: Detection of Activated Immune Cells in In Vitro Blood Model

### 1.1 Detection of Activated Immune Cells in E.coli-Infected Blood Model

In order to examine whether immune cells activated by E. coli infection may be detected by using magnetic particles, blood and E.coli were mixed to activate immune cells in vitro, and immune cells that interacted with magnetic particles were detected to evaluate a degree of activation of the immune cells in the blood, as in the following experiments.

10⁴ CFU/mL, 10⁶ CFU/mL, or 10⁶ CFU/mL of E.coli was injected into the whole blood which was collected from the tail of a male rat (Wistar rat) weighing 400 g, respectively to prepare in vitro infection blood models. Mannose-binding lectin (10405-HNAS, Sino Biological Inc., China) was immobilized on the surface of magnetic particles (03122, Ademtech, France) having a diameter of 200 nm, and these particles were mixed with the infection blood model at a concentration of 0.2 mg/mL and 5 mM calcium chloride, and allowed to react at 37° C. for 20 minutes.

A portion of the infection blood reacted with the magnetic particles was mixed with an ACK lysis buffer (Thermo Fisher Scientific, USA) at a ratio of the buffer: the infection blood model=10:1, and then mixed with 1%(w/v) DAPI (D9542, Sigma-Aldrich, USA), and 1% tween 20, and allowed to react for 30 minutes to stain immune cells, e.g., white blood cells. Thereafter, 10 μL thereof was collected and put in a cytometer to count the number of cells.

The rest of the infection blood reacted with the magnetic particles was put in a 1.5 mL EP tube, and then one surface of the tube was put to a magnet to apply a magnetic field for 20 minutes. 20 minutes later, the blood in the EP tube was carefully pipetted with saline to be washed twice while fixing the blood model to the magnet. Then, staining of the white blood cells with DAPI was performed in the same manner as above. After staining, the fixed magnet was removed, and the magnetic particles and white blood cells containing the magnetic particles which were induced on the surface inside the tube, where the magnet had been placed, were well suspended in a saline solution, and 10 μL thereof was put in the cytometer to count the number of cells.

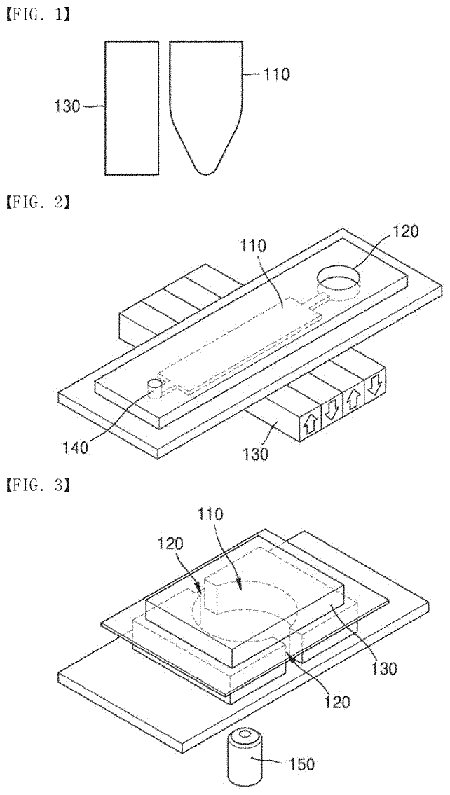

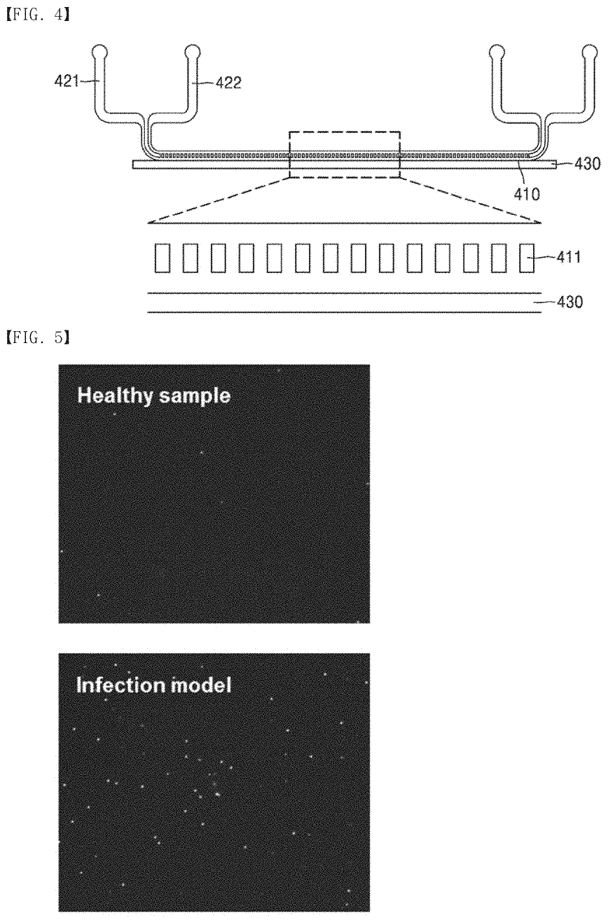

These procedures were performed in the apparatus of FIG. 1, and blood which was not mixed with E.coli was used as a control group. The control group and the infection blood model were photographed under a fluorescence microscope, and shown in FIG. 5.

As shown in FIG. 5, it was confirmed that the number of the immune cells which were isolated by the magnetic field by interacting with the magnetic particles was significantly increased in the infection blood model (infection model), as compared with the control group (healthy sample).

Further, the blood model infected with 10⁴ CFU/mL of E.coli showed about 1.45% increase in the number of the immune cells that were pulled toward the magnet by the magnetic field by including more magnetic particles, as compared with the control group. The blood model infected with 10⁶ CFU/mL of E.coli showed about 6.5% increase in the number of the immune cells that were pulled toward the magnet, as compared with the control group. The blood model infected with 10⁸ CFU/mL of E. coli showed about 57.10% increase in the number of the immune cells that were pulled toward the magnet, as compared with the control group.

As a result, the immune cells in the blood were activated in proportion to the concentration of infected E. coli, and a larger number of immune cells included magnetic particles via endocytosis of activated immune cells, suggesting that the degree of infection may be predicted by the above method.

### 1.2 Detection of Activated Immune Cells in Lipopolysaccharide (LPS)-Infected Blood Model

In order to examine whether immune cells activated by LPS infection may be detected by using magnetic particles, blood was mixed with 1 mg/mL of LPS, and experiments were performed in the same manner as in 1.1.

As an experimental result, the LPS-infected blood showed about 14.37% increase in the number of white blood cells that were pulled toward the magnet, as compared with the control group.

As a result, a larger number of magnetic particles interacted with the immune cells via endocytosis of the immune cells activated by LPS infection, suggesting that the number of activated immune cells or a degree of activation of immune cells may be diagnosed by the above method and apparatus,

### Example 2: Detection of Activated Immune Cells in In Vivo Rat Model

### 2.1 Detection of Activated Immune Cells in E.coli-Infected Rat Model

In order to examine whether activation of immune cells may be detected in infected rats, E.coli was injected into rats to activate immune cells, and immune cells that interacted with the magnetic particles were detected to evaluate a degree of activation of the immune cells, as in the following experiments.

10⁷ CFU/mL of E.coli was added to 1 mL of saline solution, and this solution was administered to a male rat (Wistar rat) weighing 400 g by intraperitoneal injection to prepare an infected rat model. Before infection and 4 hrs after infection, whole blood was collected from the tail. The whole blood was mixed with magnetic particles having a diameter of 200 nm, wherein mannose-binding lectin was immobilized on the surface of magnetic particles, at a concentration of 0.2 mg/mL and 5 mM calcium chloride, and allowed to react at 37° C. for 20 minutes. After reaction, in order to measure immune cells in the blood, 5 μM of cell tracker (Molecular Probes Life technologies, USA) was added to an ACK lysis buffer (Thermo Fisher Scientific, USA), and fluorescent staining of the immune cells was performed for 20 minutes. After fluorescent staining, the blood was left as it is, and immune particles were allowed to settle down. Immune cells that settled down were detected by a fluorescence microscope.

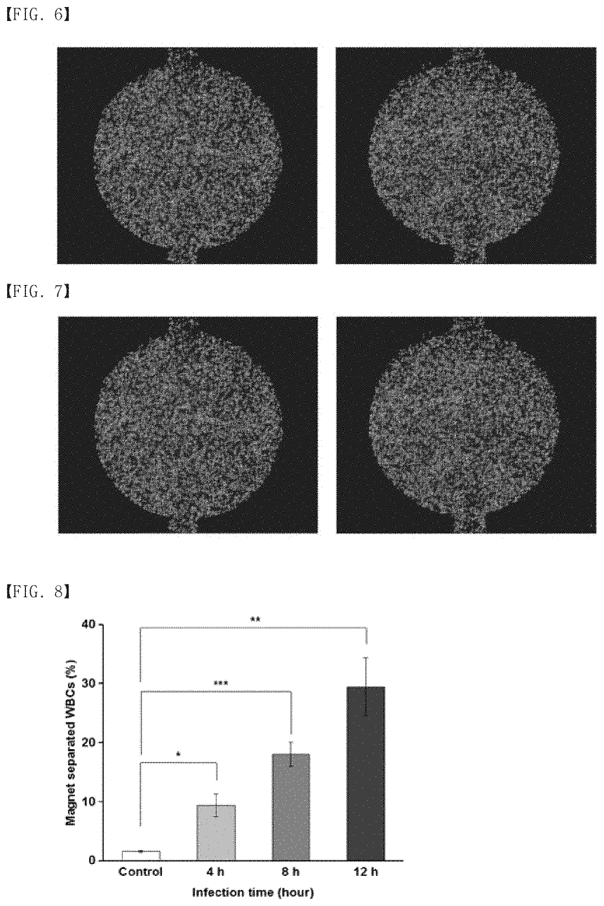

After detection, a magnet was used to apply a magnetic field and immune cells remaining after separating the immune cells that interacted with the magnetic particles were allowed to settle down and detected by using a fluorescence microscope (ImageJ, USA). These procedures took place in the apparatus shown in FIG. 3, and all blood collected prior to administration of E.coli to the rat was used as a control group. The experimental result of the control group is shown in FIG. 6, and the experimental result of the infected rat model is shown in FIG. 7.

FIG. 6 shows a photograph (left) of immune cells before contacting the blood with magnetic particles and a photograph (right) of remaining immune cells except for immune cells that interacted with magnetic particles by contacting, in the case of the control rat.

FIG. 7 shows a photograph (left) of immune cells before contacting the blood with magnetic particles and a photograph (right) of remaining immune cells except for immune cells that interacted with magnetic particles by contacting, in the case of the E.coli-infected rat.

As shown in FIG. 6, the control group showed no difference in the number of immune cells before and after contacting with magnetic particles.

As shown in FIG. 7, the infected rat showed a significant decrease in the number of remaining immune cells except for immune cells that interacted with magnetic particles after contacting with the magnetic particles (about 24% of immune cells were reacted with the magnetic particles, as compared with the immune cells before contacting with the magnetic particles).

In the case of the infected rat, immune cells in the blood were activated by E.coli, and magnetic particles that interacted with the immune cells were increased by active endocytosis of the activated immune cells, and therefore, it was confirmed that a degree of activation of immune cells may be evaluated in vivo by the method and apparatus of the present disclosure.

Further, experiments for examining the number of white blood cells according to the infection time were performed as follows.

10⁸ CFU/mL of E. coli-K12 was prepared in 1 mL of a physiological saline solution, which was then intraperitoneally injected into 400 g of 8-week-old male wistar rat to prepare a sepsis model. At 4 hrs, 8 hrs, and 12 hrs post-injection of E. coli-K12, blood was collected from the sepsis rat model to obtain the whole blood. Mannose binding lectin (MBL)-imrnobilized magnetic particles with a diameter of 200 nm were mixed with the whole blood, and allowed to react at room temperature for 1 hr, and then the number of cells drawn toward a magnet was compared. When the white blood cells were counted, 1% DAPI (Sigma, USA) and Tween 20 (Sigma, USA) in an ACK lysis buffer (Thermo Fisher Scientific, USA) or a physiological saline solution were prepared, and only the white blood cells were fluorescence-stained for 20 minutes.

As a result, about 3% of the total number of the white blood cells in a control group were drawn toward the magnet due to the effect of the magnetic field by including magnetic particles, and about 10% in an experimental group at 4 hours post-sepsis, about 20% in an experimental group at 8 hours post-sepsis, and about 30% in an experimental group at 12 hours post-sepsis, indicating that endocytosis was increased over time after infection (FIG. 8).

Therefore, in infected rats, immune cells in the blood were activated by E.coli, and magnetic particles that interacted with the immune cells were increased by active endocytosis of the activated immune cells, indicating that a degree of activation of immune cells in in-vivo rat model may be evaluated by the method and the apparatus of the present disclosure.

FIG. 8 is a graph showing white blood cells detected at each time point of infection of rats with E. coli, through the apparatus for isolating activated immune cells according to an embodiment.

### Example 3: Comparison of the Present Disclosure with Existing Method of Diagnosing Sepsis

To compare the present disclosure with an existing method of diagnosing sepsis, the control group and the blood samples at 4 hrs, 8 hrs, and 12 his post-infection which were used in the experiment of Examples 2.1 were injected into a blood culture (BACTEC™), respectively, followed by incubation at 37°°C. for 7 days. After 7 day-incubation, to examine the presence or absence of bacteria, bacteria were examined by an agar plating method, and as a result, negative results were observed in all the conditions of the control group and the blood samples at 4 hrs, 8 hrs, and 12 hrs post-infection. For a positive control, organs were removed from the rat at 12 hrs post-infection, and the presence or absence of E. coli in the organs was examined by immunofluorescence (IF) method.

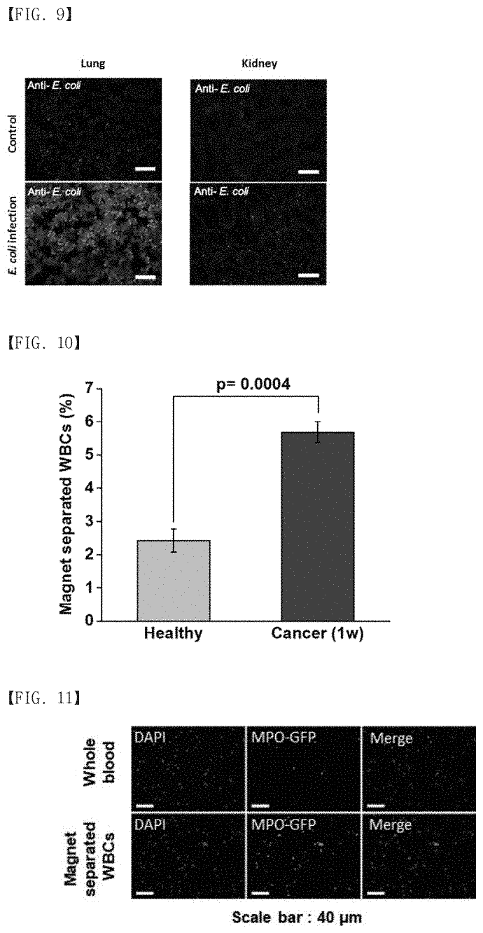

As a result, more fluorescent signals of E. coli were detected in the organs (lung and kidney) at 12 hrs post-infection than in the control organs (FIG. 9). In the existing bacteria blood culture method, negative results were observed even at 12 hrs post-infection, whereas the present disclosure showed the obvious difference between the control group and the infected group, indicating that the present disclosure may be a more effective diagnostic method than the existing method of diagnosing sepsis.

FIG. 9 is an immunofluorescence image (IF) of identifying the presence of E.coli in the organ of the rat.

### Example 4: Detection of Activated Immune Cells in Breast Cancer Mouse Model

To examine whether activated immune cells are able to be detected in a breast cancer mouse model, a degree of activation of immune cells were evaluated by detecting magnetic particles that interacted with activated immune cells, as follows.

3×10⁶ cells/mL of a breast cancer cell line 4T1 was prepared in 1 mL of PBS, and injected into a mammary fat pad of 20 g of 8-week-old female balb/c, mouse to prepare a breast cancer model. 1 week later, the blood was collected from a control mouse into which cancer cells were not injected, and an cancer model experimental mouse group into which cancer cells were injected, thereby obtaining the whole blood, respectively. Mannose binding lectin (MBL)-immobilized magnetic particles with a diameter of 200 nm were mixed with the whole blood, and allowed to react at room temperature for 1 hr, and then the number of cells drawn toward a magnet was compared. When the white blood cells were counted, 1% DAPI (Sigma, USA) and Tween 20 (Sigma, USA) in an ACK lysis buffer (Thermo Fisher Scientific, USA) or a physiological saline solution were prepared, and only the white blood cells were fluorescence-stained for 20 minutes.

As a result, in the whole blood of the control group, about 2.5% of the total number of the white blood cells were drawn toward the magnet clue to the effect of the magnetic field by including magnetic particles. In the breast cancer model experiment, about 5.5% of the total number of the white blood cells were drawn toward the magnet due to the effect of the magnetic field by including magnetic particles, indicating a significant difference between the control group and the cancer model group (FIG. 10).

Therefore, immune cells in the blood were activated by cancer cells, and the immune cells that interacted with magnetic particles were increased by active endocytosis of the activated immune cells, indicating that a degree of activation of immune cells in the cancer model may be evaluated by the method and the apparatus of the present disclosure.

FIG. 10 is a graph showing white blood cells detected in the breast cancer mouse model, through the apparatus for isolating activated immune cells according to an embodiment.

### Example 5: Identification of Type of White Blood Cells Interacting with MBL-Immobilized Magnetic Nanoparticles

To identify the type of white blood cells that reacted with MBL-immobilized magnetic nanoparticles, among various types of white blood cells present in the blood, such as monocytes, lymphocytes, neutrophils, basophils, etc., cell fluorescence staining was performed using a myeloperoxidase (MPO) antibody which is a neutrophil marker, as follows.

MBL-immobilized magnetic nanoparticles were added to the blood of the rat, followed by mixing for 20 minutes, and then white blood cells bound to magnetic nanoparticles were isolated using a magnet. Cell fluorescence staining was performed for 24 hrs using DAPI and anti-MPO antibody, and images were obtained using a fluorescent microscope (FIG. 11).

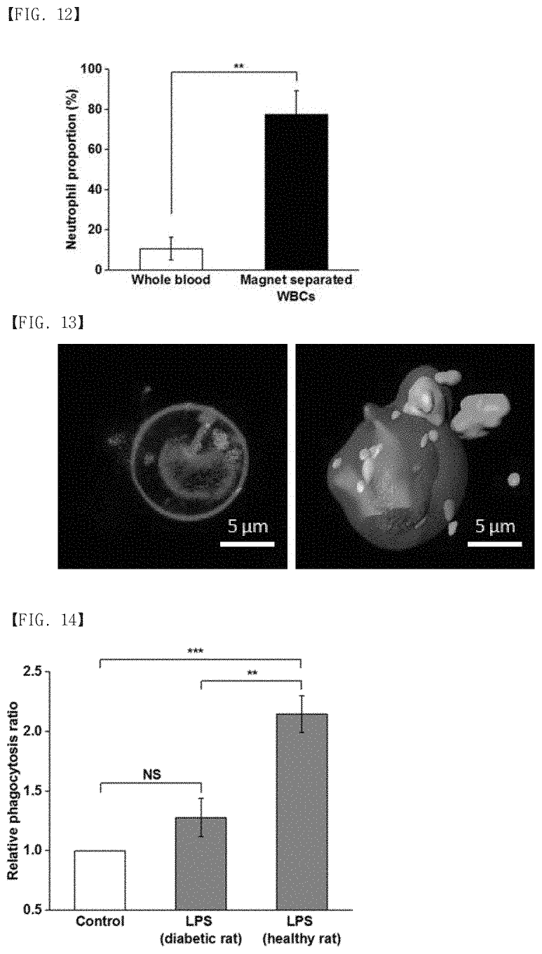

As a result, in the whole blood before treated with magnetic nanoparticles, neutrophils occupied about 10% of the total white blood cells, and in the white blood cells bound to magnetic nanoparticles and drawn toward the magnet, neutrophils occupied about 80% (FIG. 12).

Therefore, most of the white blood cells that interacted with MBL-immobilized magnetic nanoparticles were neutrophils.

FIG. 11 is an image of fluorescence-stained white blood cells, through the apparatus for isolating activated immune cells according to an embodiment.

FIG. 12 is a graph showing neutrophils in the white blood cells which were isolated using magnetic particles, through the apparatus for isolating activated immune cells according to an embodiment.

### Example 6: Analysis of Confocal Image of White Blood Cells Reacting with MBL-Immobilized Magnetic Nanoparticles

To analyze confocal images of white blood cells reacting with MBL-immobilized magnetic nanoparticles, DAPI, GFP, and Dil fluorescence staining method was used to perform cell fluorescence staining of the nuclei of white blood cells, MBL-magnetic nanoparticles, and cell surface membrane of white blood cells, as follows.

MBL-immobilized magnetic nanoparticles were added to the blood of the rat, followed by mixing for 20 minutes, and then white blood cells bound to magnetic nanoparticles were isolated using a magnet. Thereafter, DAPI, GFP fluorescence-emitting MBL-magnetic nanoparticles, and Dil were used to perform cell fluorescence staining for 24 hrs, and then images were obtained using a fluorescence microscope (FIG. 13).

As a result, it was observed that the white blood cells recognized MBL, and thus MBL-magnetic nanoparticles (green in FIG. 13) were bound to the surface of white blood cells (red in FIG. 13) or endocytosis into the cells occurred.

FIG. 13 is a fluorescence microscopy image of white blood cells reacted with MBL-immobilized magnetic particles.

### Example 7: Measurement of In-vitro Immune Activity Against External Stimulant in Normal and Diabetic Model Rats

To measure a degree of activation of immune cells when an external stimulant enters the body, lipopolysaccharide (LPS) was injected into the whole blood of a normal rat and the whole blood of a diabetic rat, respectively, and as a control group, a physiological saline solution was injected into each case, respectively. The increase rate of endocytosis was compared, as follows.

The physiological saline solution or 3 μg/mL of LPS was injected into the whole blood collected from the rats, followed by mixing at 37° C. for 1 hr. The samples completely mixed were mixed with 0.2 mg/mL of MBL-immobilized magnetic nanoparticles and calcium chloride (5 mM), and allowed to react at 37° C. for 20 minutes. Then, the number of cells drawn toward a magnet was compared. When the white blood cells were counted, 1% DAPI (Sigma, USA) and Tween 20 (Sigma, USA) in an ACK lysis buffer (Thermo Fisher Scientific, USA) or the physiological saline solution were prepared, and only the white blood cells were fluorescence-stained for 20 minutes.

As a result, with regard to the whole blood of the healthy rat, endocytosis was increased twice or more in the LPS-injected experimental group, as compared with the non-LPS-injected control group. There was no significant difference in endocytosis between the LPS-injected experimental group and the non-LPS-injected control group in the diabetic rat model suspected of having reduced immune function (FIG. 14).

Therefore, the present disclosure may quantitatively determine immune functions of immune cells by measuring the degree of activation of immune cells of an individual against external stimulation, thereby being used in diagnosing immune function-related diseases.

FIG. 14 is a graph showing a comparison of endocytosis between the diabetic rat model and the normal rat, through the apparatus for isolating activated immune cells according to an embodiment.

1. A method of evaluating a degree of activation of immune cells of a subject, the method comprising:
contacting magnetic particles with a sample separated from the subject, wherein the sample comprises the immune cells;
isolating immune cells that interacted with the magnetic particles from the immune cells in the sample by applying a magnetic field to a reaction product obtained by the contacting; and
detecting the isolated immune cells that interacted with the magnetic particles.

2. The method of claim 1, wherein the sample is selected from the group consisting of blood, urine, feces, saliva, lymph, cerebrospinal fluid, synovial fluid, cystic fluid, ascites, interstitial fluid, and ocular fluid.

3. The method of claim 1, wherein the immune cells comprise neutrophils, eosinophils, basophils, monocytes, lymphocytes, macrophages, mast cells, B cells, T cells, natural killer cells, or any combination thereof.

4. The method of claim 1, wherein the magnetic particles are oxidized or surface-modified with metals, functional groups, proteins, carbohydrates, polymers, or lipids.

5. The method of claim 1, wherein the contacting is performed at 0° C. to 40° C. for 0.1 second to 1 hour.

6. The method of claim 1, wherein the applying of the magnetic field is performed for 0.1 second to 1 hour.

7. The method of claim 1, wherein the immune cells that interacted with the magnetic particles interact with the magnetic particles via endocytosis of the immune cells.

8. The method of claim 1, wherein the method of evaluating the degree of activation of the immune cells of the subject further comprises:
comparing a level of the detected immune cells (activated immune cells) that interacted with the magnetic particles with a level of the immune cells in the sample, or comparing the level of the immune cells that interacted with the magnetic particles in the sample separated from the subject with a level of immune cells that interacted with magnetic particles in a sample separated from a normal subject.

9. A method of diagnosing immune-related diseases, the method comprising:
contacting magnetic particles with a sample separated from a subject, wherein the sample comprises immune cells;
isolating immune cells that interacted with the magnetic particles from the immune cells in the sample by applying a magnetic field to a reaction product obtained by the contacting; and
detecting the isolated immune cells that interacted with the magnetic particles.

10. An apparatus for separating activated immune cells, the apparatus comprising:
a chamber for storing a sample comprising immune cells and magnetic particles; and
a magnetic field-forming portion which is disposed to apply a magnetic field around the chamber, wherein activated immune cells, among the immune cells in the sample, interact with magnetic particles to form magnetic particle-immune cell complexes, and the magnetic particle-immune cell complexes are collected around a magnetic field formed by the magnetic field-forming portion.

11. The apparatus of claim 10, further comprising an inlet which is connected to an end part of the chamber.

12. The apparatus of claim 10, further comprising an outlet which is connected to another end part of the chamber.

13. The apparatus of claim 10, further comprising a detecting portion for detecting magnetic particle-immune cell complexes.

14. The apparatus of claim 10, wherein the chamber comprises one or more selected from the group consisting of a tube, a channel, a droplet, and a well.

15. The apparatus of claim 10, wherein the magnetic field-forming portion comprises one or more magnets.

16. The apparatus of claim 10, wherein the sample is selected from the group consisting of blood, urine, feces, saliva, lymph, cerebrospinal fluid, synovial fluid, cystic fluid, ascites, interstitial fluid, and ocular fluid, all of them being separated from a subject.

17. The apparatus of claim 10, wherein the magnetic particles have a diameter of 1 nm to 30 μm.

18. The apparatus of claim 10, further comprising a plurality of inlets connected to an end part of the chamber, wherein the chamber further comprises a separating portion, and the magnetic field-forming portion is disposed on a surface of the chamber to apply a magnetic field.
